(19) United States
(12) Patent Application Publication
(10) Pub. No.: US 2024/0428906 A1
(43) Pub. Date: Dec. 26, 2024

(54) METHOD AND APPARATUS USING COMPUTATIONAL PATHOLOGY FOR RISK STRATIFICATION OF CANCER

(71) Applicants: Case Western Reserve University, Cleveland, OH (US); Emory University, Atlanta, GA (US); The United States Government as Represented by The Department of Veteran Affairs, Washington, DC (US)

(72) Inventors: Yuli Chen, Xi'an (CN); Haojia Li, Cleveland, OH (US); Anant Madabhushi, Decatur, GA (US)

(21) Appl. No.: 18/651,738

(22) Filed: May 1, 2024

Related U.S. Application Data
(60) Provisional application No. 63/579,059, filed on Aug. 28, 2023.
(60) Provisional application No. 63/509,540, filed on Jun. 22, 2023.

(51) Int. Cl. G16H 10/40; G16H 50/30

(52) U.S. Cl. CPC G16H 10/40; G16H 50/30

(57) ABSTRACT

The present disclosure relate to a method. The method includes accessing segmented digitized pathology imaging data from a cancer patient. The segmented digitized pathology imaging data identifies segmented nuclei, segmented mitosis, and segmented tubule regions. A plurality of nuclear features are extracted using the segmented nuclei. A plurality of mitosis features are extracted using the segmented mitosis. A plurality of tubule features are extracted using the segmented tubule regions. A risk score is generated by operating a machine learning model on the plurality of nuclear features, the plurality of mitosis features, and the plurality of tubule features. The risk score correlates to a risk of recurrence of cancer for the cancer patient.

### REFERENCE TO RELATED APPLICATION

This application claims the benefit of U.S. Provisional Application No. 63/509,540, filed on Jun. 22, 2023, the contents of which are hereby incorporated by reference in their entirety. This application also claims the benefit of U.S. Provisional Application No. 63/579,059, filed on Aug. 28, 2023, the contents of which are hereby incorporated by reference in their entirety.

### FEDERAL FUNDING INFORMATION

This invention was made with government support under CA202752 awarded by the National Institutes of Health. The government has certain rights in the invention.

### BACKGROUND

The breast is an organ found in humans that sits on top of the upper ribs and chest muscles. Breast cancer is a cancer that forms in cells of the breast. Breast cancer occurs almost entirely in women. Over time, it is common for breast cancer to spread into surrounding tissue. Such breast cancer is known as invasive breast cancer.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates a block diagram of some embodiments of a cancer risk assessment system configured to determine risk stratifications using nuclear features, mitosis features, and tubule features.

FIG. 2 illustrates a block diagram of some additional embodiments of a disclosed cancer risk assessment system.

FIG. 3 illustrates exemplary Kaplan-Meier curves associated with a disclosed cancer risk assessment system.

FIG. 4A illustrates a block diagram of some embodiments of a disclosed breast cancer risk assessment system that is configured to operate in conjunction with other assessment methods to provide improved risk stratification.

FIG. 4B illustrates exemplary Kaplan-Meier curves showing a probability of survival as a function of time for a disclosed breast cancer risk assessment system applied to different classifications of Oncotype Dx.

FIG. 4C illustrates exemplary Kaplan-Meier curves showing a probability of survival as a function of time for a disclosed breast cancer risk assessment system applied to different classifications of a Nottingham grading system (NGS).

FIG. 5 illustrates a block diagram of some additional embodiments of a disclosed breast cancer risk assessment system.

FIG. 6A illustrates a table showing exemplary nuclear, mitotic, and tubule formation features and associated coefficients of a prognostic Cox-regression model.

FIG. 6B illustrates a table showing exemplary nuclear, mitotic, and tubule formation features.

FIG. 7 illustrates exemplary violin graphs showing feature values of different nuclear, mitotic, and tubule features for high-risk and low-risk patients.

FIG. 8 illustrates exemplary whole slide images showing exemplary nuclear, mitotic, and tubule features.

FIG. 9 shows a block diagram illustrating exemplary training of a disclosed breast cancer risk assessment system.

FIG. 10 illustrates a block diagram of some additional embodiments of a disclosed breast cancer risk assessment system.

FIG. 11 illustrates a flow diagram of some embodiments of a method of assessing cancer risk using nuclear, mitotic, and tubule features extracted from digitized pathology imaging data.

FIG. 12 illustrates a block diagram of some embodiments of a disclosed cancer risk assessment system.

FIG. 13 illustrates a table showing clinical variables of patient cohorts used in an example use case of the disclosed breast cancer risk assessment system.

FIG. 14 illustrates exemplary Kaplan-Meier curves showing a probability of survival as a function of time in the example use case of the disclosed breast cancer risk assessment system.

FIG. 15 illustrates a table showing univariate and multivariable analysis of disease-free survival for the patient cohorts used in the example use case of the disclosed breast cancer risk assessment system.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Breast cancer is the most frequently diagnosed cancer and the second leading cause of cancer-related death for females worldwide. A common subtype of invasive breast cancer is estrogen receptor-positive and lymph node-negative invasive breast cancer (ER+ & LN− IBC). A standard treatment for ER+ & LN− IBC includes breast-conserving surgery (e.g., a partial mastectomy that removes only the tumor and a margin of surrounding healthy tissue) followed by radiation, adjuvant chemotherapy, and/or adjuvant hormonal therapy. Given the significant side effects of adjuvant chemotherapy, it is ideally adopted only for patients that are at a high-risk of recurrence. Therefore, it is desirable to identify ER+ & LN− IBC patients with low risk of recurrence who may safely avoid adjuvant chemotherapy.

Oncotype Dx (ODx) is a widely applied and extensively validated molecular assay in clinical practice, with ODx score aiding in estimating a risk of ER+ & LN− IBC recurrence and a treatment benefit from adjuvant chemotherapy. The ODx test is, however, expensive and usually tissue destructive. Furthermore, it has been appreciated that the ODx risk classifications may not always be accurate. For example, ODx risk classifications may be less accurate on African American patients as compared to Caucasian patients. In addition, some patients identified as being in one ODx risk classification might actually have a risk of recurrence associated with a different ODx risk classification.

The Nottingham grading system (NGS) is also used by pathologists to evaluate a risk of recurrence in patients having ER+ & LN− IBC. The NGS consists of a visual assessment that uses three components: 1) nuclear pleomorphism referring to variations in nuclear shape, size, and chromatin appearance, 2) mitotic activity relating to tumor cell division and proliferation, and 3) tubule formation reflecting a percentage of tumor cells forming tubule structures. However, NGC fails to provide good subjectivity and inter-observer variability in clinical practice.

The present disclosure relates to a method and apparatus configured to generate a risk score for cancer patients by applying a regression model to extracted nuclear features, mitosis features, and tubule features. In some embodiments, the method may comprise accessing automatically segmented data from one or more digitized pathology images of a cancer patient. The automatically segmented data identifies segmented nuclei, segmented mitosis, and segmented tubule regions. A plurality of nuclear features are extracted from the segmented nuclei, a plurality of mitosis features are extracted from the segmented mitosis, and a plurality of tubule features are extracted from the segmented tubule regions. A risk score is generated by operating a regression model on the plurality of nuclear features, the plurality of mitosis features, and the plurality of tubule features. The risk score correlates to a risk of recurrence for the cancer patient (e.g., a likelihood of disease-free survival). By using features extracted from automatically segmented nuclei, mitosis, and tubule regions, the disclosed method is able to provide for a relatively low-cost, fast, and highly reproducible risk score that is able to accurately predict a risk of recurrence of cancer (e.g., breast cancer) while mitigating inter-observer variability.

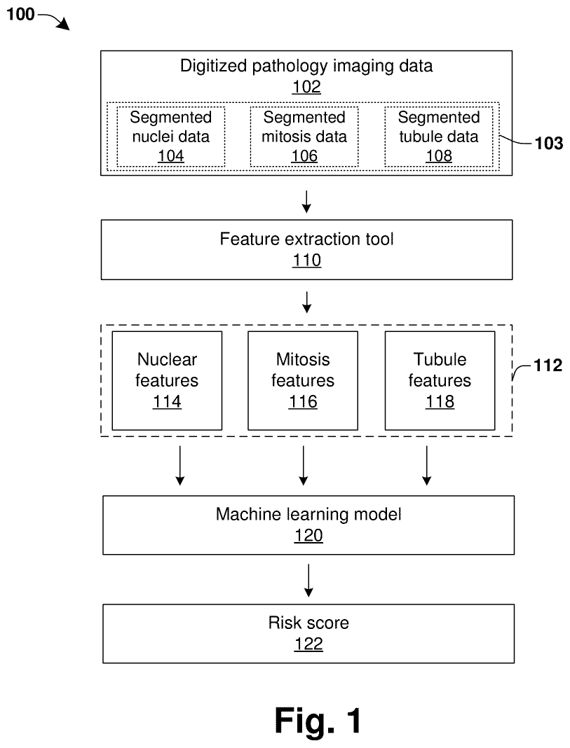

FIG. 1 illustrates a block diagram of some embodiments of a cancer risk assessment system configured to determine risk stratifications using nuclear features, mitosis features, and tubule features.

The cancer risk assessment system 100 comprises a feature extraction tool 110 that is configured to access digitized pathology imaging data 102 corresponding to a cancer patient (e.g., a patient that has had, that currently has, and/or that is suspected of having cancer). The digitized pathology imaging data 102 may comprise segmented digitized pathology imaging data 103. The segmented digitized pathology imaging data 103 may include automatically segmented nuclei data 104 that identifies segmented nuclei, automatically segmented mitosis data 106 that identifies segmented mitosis, and automatically segmented tubule data 108 that identifies segmented tubule regions within a digitized pathology image of the cancer patient. In some embodiments, the cancer patient may have had or currently have estrogen receptor-positive and lymph node-negative invasive breast cancer (ER+ & LN− IBC) or estrogen receptor-positive and lymph node-positive invasive breast cancer (ER+ & LN+ IBC). In other embodiments, the cancer patient may have had or currently have another type of cancer (e.g., colon cancer, pancreatic cancer, etc.) that has tubule structures.

The feature extraction tool 110 is configured to extract a plurality of features 112 (e.g., hand-crafted features) from the segmented digitized pathology imaging data 103. The plurality of features 112 include a plurality of nuclear features 114 (e.g., nuclear morphology features, nuclear histomorphometric features, and/or the like), a plurality of mitosis features 116, and a plurality of tubule features 118. In some embodiments, the feature extraction tool 110 is configured to extract the plurality of nuclear features 114 using the automatically segmented nuclei data 104, the plurality of mitosis features 116 using the automatically segmented mitosis data 106, and the plurality of tubule features 118 using the automatically segmented tubule data 108.

A machine learning model 120 is configured to operate upon the plurality of nuclear features 114, the plurality of mitosis features 116, and the plurality of tubule features 118 to generate a risk score 122. In some embodiments, the risk score 122 is indicative of the cancer patient's risk of recurrence for cancer (e.g., breast cancer, pancreatic cancer, colon cancer, etc.) and/or a likelihood of disease-free survival. By operating the machine learning model 120 upon the plurality of nuclear features 114, the plurality of mitosis features 116, and the plurality of tubule features 118, the disclosed cancer risk assessment system 100 is able to generate the risk score 122 to be significantly prognostic of disease-free survival. For example, the disclosed cancer risk assessment system 100 is able to generate the risk score 122 for breast cancer having a hazard ratio (HR) of 2.94 (95% CI=1.18-7.35, p=0.0208) on validation data sets.

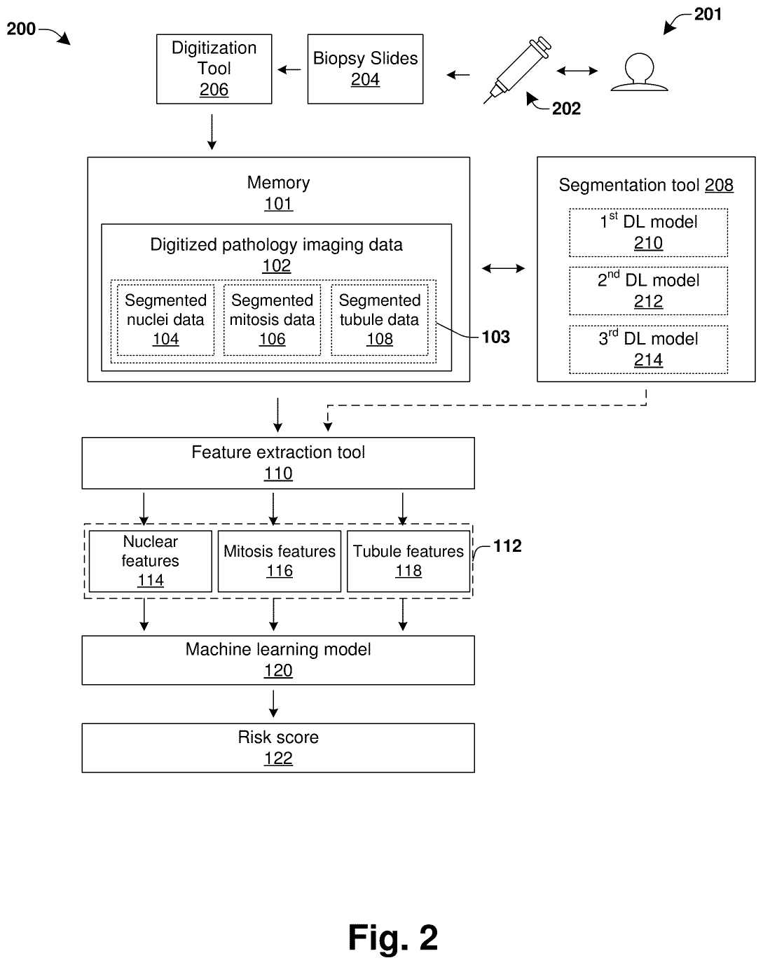

FIG. 2 illustrates a block diagram of some additional embodiments of a disclosed cancer risk assessment system 200.

The cancer risk assessment system 200 comprises a memory 101 configured to store digitized pathology imaging data 102. The digitized pathology imaging data 102 comprises one or more digitized pathology images from a cancer patient 201. In some embodiments, the cancer patient 201 may have breast cancer (e.g., estrogen receptor-positive and lymph node-negative invasive breast cancer (ER+ & LN− IBC)). In some embodiments, the one or more digitized pathology images may comprise a whole slide image (WSI), a patch of a WSI, or the like. In some embodiments, the memory 101 may comprise electronic memory (e.g., solid state memory, SRAM (static random-access memory), DRAM (dynamic random-access memory), and/or the like).

In some embodiments, the digitized pathology imaging data 102 may be generated from a pathological tissue sample taken from the cancer patient 201. In some such embodiments, a tissue sample collection tool 202 (e.g., a cannular, forceps, needle, punch, scalpel, and/or the like) is used to excise tissue from the cancer patient 201 to obtain a tissue block. The tissue block is fixed, embedded in paraffin, and sliced into thin slices (e.g., using a microtome), which are subsequently placed on one or more transparent slides (e.g., one or more glass slides). The tissue on the one or more transparent slides is then stained to generate one or more biopsy slides 204. A slide digitization element 206 is used to convert the one or more biopsy slides 204 to a plurality of whole slide images (WSIs), which are stored as digitized biopsy images in the memory 101 as part of the digitized pathology imaging data 102 (e.g., comprising an CMOS image sensor, a CCD camera, and/or the like). In some embodiments, the one or more digitized pathology images may comprise a digitized H&E (Hematoxylin and Eosin) stained slide.

A segmentation tool 208 comprising a plurality of deep learning segmentation models 210-214 is configured to access the digitized pathology imaging data 102. The plurality of deep learning segmentation models 210-214 are configured to segment the digitized pathology imaging data 102 to identify nuclei, mitosis, and tubule regions. In some embodiments, the plurality of deep learning segmentation models 210-214 are configured to generate segmented digitized pathology imaging data 103 including automatically segmented nuclei data 104 that identifies the nuclei, automatically segmented mitosis data 106 that identifies the mitosis, and automatically segmented tubule data 108 that identifies the tubule regions. In some embodiments, the automatically segmented nuclei data 104, the automatically segmented mitosis data 106, and the automatically segmented tubule data 108 may comprise or be binary masks. In some such embodiments, the one or more binary masks may comprise or be images having a value of “1” in image units (e.g., pixels, voxels, etc.) identified as being within a nuclei, mitosis, and/or tubule region and having a value of “0” in image units outside of the nuclei, mitosis, and/or tubule regions. In some embodiments, the segmented digitized pathology imaging data 103 may be saved back to the memory 101 as part of the digitized pathology imaging data 102.

In some embodiments, the plurality of deep learning segmentation models 210-214 may comprise a first deep learning segmentation model 210 that is configured to operate upon the digitized pathology imaging data 102 to generate the automatically segmented nuclei data 104, a second deep learning segmentation model 212 that is configured to operate upon the digitized pathology imaging data 102 to generate the automatically segmented mitosis data 106, and a third deep learning segmentation model 214 that is configured to operate upon the digitized pathology imaging data 102 to generate the automatically segmented tubule data 108. It has been appreciated that using different deep learning segmentation models to generate the segmented digitized pathology imaging data 103 allows for segmentation to be performed to a sufficient accuracy to allow the cancer risk assessment system 200 to generate a risk score that has a high prognostic value. In some embodiments, the plurality of deep learning segmentation models 210-214 may be run on one or more processors (e.g., a central processing unit including one or more transistor devices configured to operate computer code to achieve a result, a microcontroller, or the like).

A feature extraction tool 110 is configured to extract a plurality of features 112 from the digitized pathology imaging data 102. The plurality of features 112 include a plurality of nuclear features 114, a plurality of mitosis features 116, and a plurality of tubule features 118. In some embodiments, the feature extraction tool 110 is configured to extract the plurality of nuclear features 114 from the automatically segmented nuclei data 104, the plurality of mitosis features 116 from the automatically segmented mitosis data 106, and the plurality of tubule features 118 from the automatically segmented tubule data 108. In some embodiments, the feature extraction tool 110 may be implemented as computer code run by a processing unit (e.g., a central processing unit including one or more transistor devices configured to operate computer code to achieve a result, a microcontroller, or the like).

In some embodiments, the plurality of features 112 comprise hand-crafted features that have biological interpretability. In some embodiments, the nuclear features 114 may comprise nuclear shape features, nuclear morphology features, nuclear histomorphometric features, and/or the like. In some embodiments, the mitosis features 116 may comprise features that describe mitotic rates. In some embodiments, the tubule features 118 may comprise features that describe a formation of tubules (e.g., tubule formation features). It will be appreciated that the nuclear features 114, the mitosis features 116, and the tubule features 118 are at a higher order or higher level than a human can resolve in the human mind or with pencil and paper. For example, a texture feature (e.g., Haralick feature) of cancer cells cannot be identified by a human.

A machine learning model 120 is configured to operate upon the plurality of nuclear features 114, the plurality of mitosis features 116, and the plurality of tubule features 118 to generate a risk score 122. The machine learning model 120 may generate the risk score 122 based on weightings of the plurality of features 112, thereby providing health care professionals with more insight into the risk of recurrence than black-box-based deep learning models. In some embodiments, the risk score 122 is indicative of the cancer patient's risk of recurrence of cancer (e.g., breast cancer) and/or disease-free survival. In some embodiments, the machine learning model 120 may comprise a Cox proportional hazards model run on one or more processors (e.g., including a graphics processing unit (GPU) or the like).

By using a plurality of features extracted from automatically segmented nuclei, mitosis, and tubule regions, the disclosed cancer risk assessment system 200 is able to provide for a relatively low-cost, fast, and highly reproducible risk score that is able to accurately predict a risk of recurrence for cancer patients. Furthermore, with the prevalence of WSI scanners, the disclosed cancer risk assessment system holds vast potential to serve as an inexpensive and faster alternative prognostic tool in low resource settings where molecular assays (e.g., ODx) may not be available.

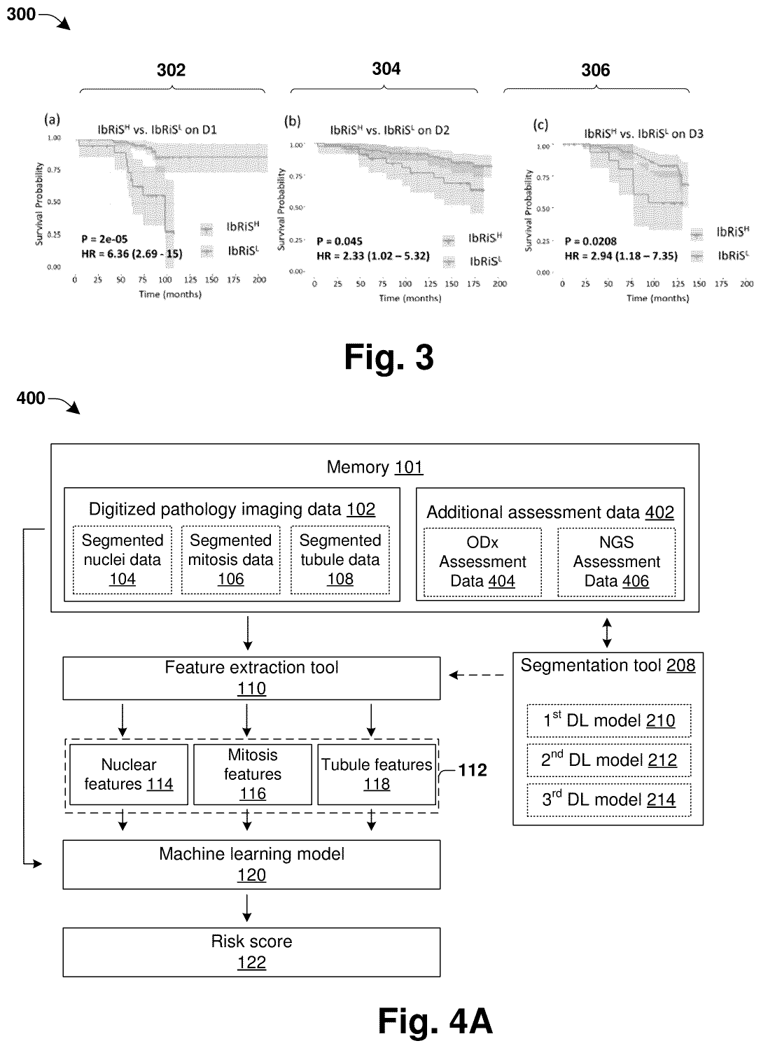

FIG. 3 illustrates exemplary Kaplan-Meier (KM) curves 300 associated with a disclosed cancer risk assessment system. The KM curves 300 show a probability of survival as a function of time for a cancer patient having breast cancer.

The KM curves 300 are shown for three different data sets 302-306. For each of the KM curves 300, the x-axis represents a time in months and y-axis represents the estimated survival probability. The KM curves 300 for the three different data sets 302-306, respectively have a high-risk stratification group (IBRiS^{H}) and a low-risk stratification group (IBRiS^{L}). In some embodiments, stratification between the high-risk stratification group and the low-risk stratification group may be based on a median of risk scores generated during training of the disclosed cancer risk assessment system.

The KM curves associated with the first data set 302 show significant differences between the high-risk stratification group (IBRiS^{H}) and the low-risk stratification group (IBRiS^{L}). The KM curves associated with the second data set 304 and the third data set 306 also show meaningful differences between the high-risk stratification groups and the low-risk stratification groups. The differences between the KM curves of the high-risk stratification groups and the low-risk stratification groups indicate that the risk scores generated by the disclosed cancer risk assessment system are highly prognostic of disease-free survival of cancer patients.

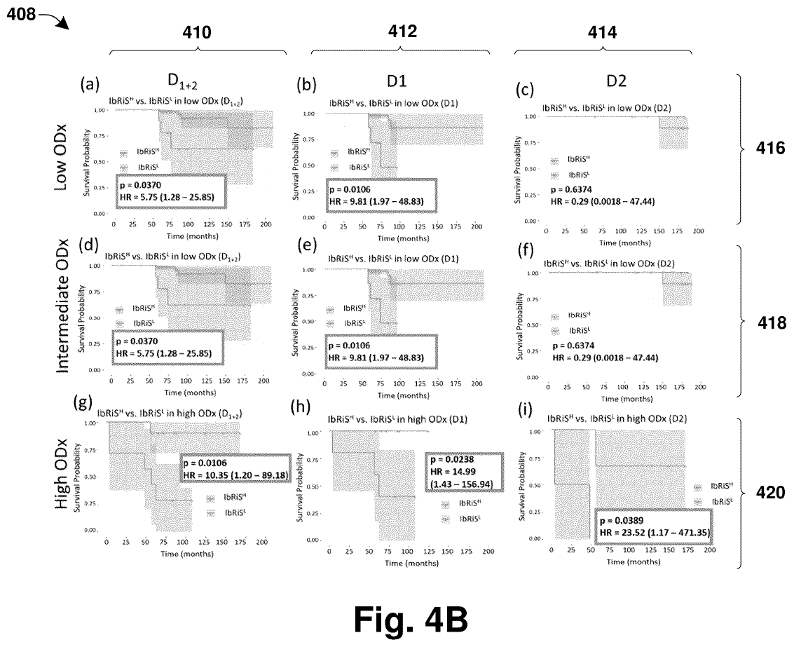

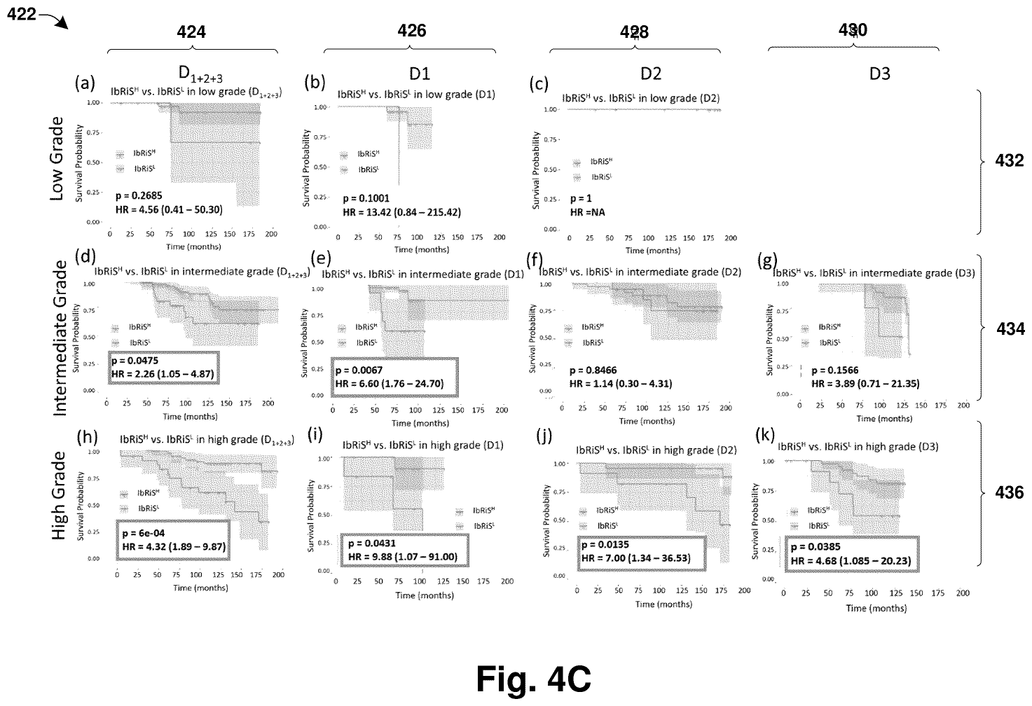

FIG. 4A illustrates a block diagram of some embodiments of a disclosed breast cancer risk assessment system 400 that is configured to operate in conjunction with other assessment methods to provide improved risk stratification.

The breast cancer risk assessment system 400 comprises a feature extraction tool 110 that is configured to extract a plurality of features 112 from digitized pathology imaging data 102 stored in a memory 101. The plurality of features 112 include a plurality of nuclear features 114, a plurality of mitosis features 116, and a plurality of tubule features 118.

Additional assessment data 402 is also stored in the memory 101. The additional assessment data 402 is associated with the digitized pathology imaging data 102 and provides for a classification of a cancer patient based on an additional assessment method. In some embodiments, the additional assessment data 402 may be an assessment of risk recurrence determined from a molecular assay (e.g., levels of Oncotype Dx (ODx) 404 in a cancer patient). For example, the additional assessment data 402 may comprise a stratification of a cancer patient into a low ODx classification that indicates a high survival probability over time, an intermediate ODx classification that indicates an intermediate survival probability over time, and a high ODx classification that indicates a low survival probability over time. In other embodiments, the additional assessment data 402 may be an assessment of risk recurrence determined a Nottingham grading system (NGS) 406. For example, the additional assessment data 402 may comprise a stratification of a cancer patient into a low NGS classification that indicates a high survival probability over time, an intermediate NGS classification that indicates an intermediate survival probability over time, and a high NGS classification that indicates a low survival probability over time.

A machine learning model 120 (e.g., a regression model) is configured to generate a risk score 122 using the plurality of features 112. The risk score 122 may be complementary to grading of the additional assessment data 402. In some embodiments, the risk score 122 may further stratify classifications provided by the additional assessment data 402. For example, the risk score 122 may further stratify a high ODx classification. In other embodiments, the risk score 122 may supplement the classifications provided by the additional assessment data 402 to help identify incorrect classifications. In some embodiments, the machine learning model 120 may use the plurality of features 112 and the additional assessment data 402 to generate the risk score 122 to be more prognostic.

The breast cancer risk assessment system 400 is able to add significant prognostic value to the ODx classification. For example, for cancer patients distributed in the high ODx classification, the breast cancer risk assessment system 400 is able to identify cancer patients with true low risk of recurrence, thus safely avoiding aggressive adjuvant chemotherapy. In some embodiments, the disclosed breast cancer risk assessment system 400 is able to achieve significant risk stratification within a high ODx classification (e.g., HR=10.35, 95% CI=1.20-89.18, p=0.0106). The high prognostic ability and good risk stratification allows disclosed breast cancer risk assessment system 400 to improved health care for cancer patients.

FIG. 4B illustrates exemplary Kaplan-Meier (KM) curves 408 showing a probability of survival as a function of time for a disclosed breast cancer risk assessment system applied to different classifications of Oncotype Dx (ODx).

The KM curves 408 are shown for three different data sets 410-414 in low ODx classifications 416, intermediate ODx classifications 418, and high ODx classifications 420. The low ODx classifications 416 correspond to cancer patients that have an ODx score that indicates a high survival probability over time. The intermediate ODx classifications 418 correspond to cancer patients that have an ODx score that indicates an intermediate survival probability over time. The high ODx classifications 420 correspond to patients that have an ODx score that indicates a low survival probability over time.

The KM curves 408 show that the disclosed breast cancer risk assessment system is able to add significant prognostic value to the ODx classifications 416-420. For example, the disclosed breast cancer risk assessment system is able to identify cancer patients with true low risk of recurrence in the high ODx classification 420. These results suggest that among the cancer patients identified as high-risk by ODx test in clinical practice, some of them, however, are in fact true low risk and could be effectively identified by the disclosed breast cancer risk assessment system, thus safely avoiding aggressive adjuvant chemotherapy.

FIG. 4C illustrates exemplary Kaplan-Meier (KM) curves 422 showing a probability of survival as a function of time for a disclosed breast cancer risk assessment system applied to different classifications of a Nottingham grading system (NGS).

The KM curves 422 are shown for four different data sets 424-430 in low grade NGS classifications 432, intermediate grade NGS classifications 434, and high grade NGS classifications 436. The low grade NGS classifications 432 correspond to cancer patients that have an NCG grade that indicates a high survival probability over time. The intermediate grade NGS classifications 434 correspond to cancer patients that have an NGS grade that indicates an intermediate survival probability over time. The high grade NGS classifications 436 correspond to cancer patients that have an NGS grade that indicates a low survival probability over time.

The KM curves 422 show that the disclosed breast cancer risk assessment system is able to stratify high-risk and low-risk cancer patients within the high histologic groups. These results suggest added prognostic value of the disclosed breast cancer risk assessment system to histologic grading.

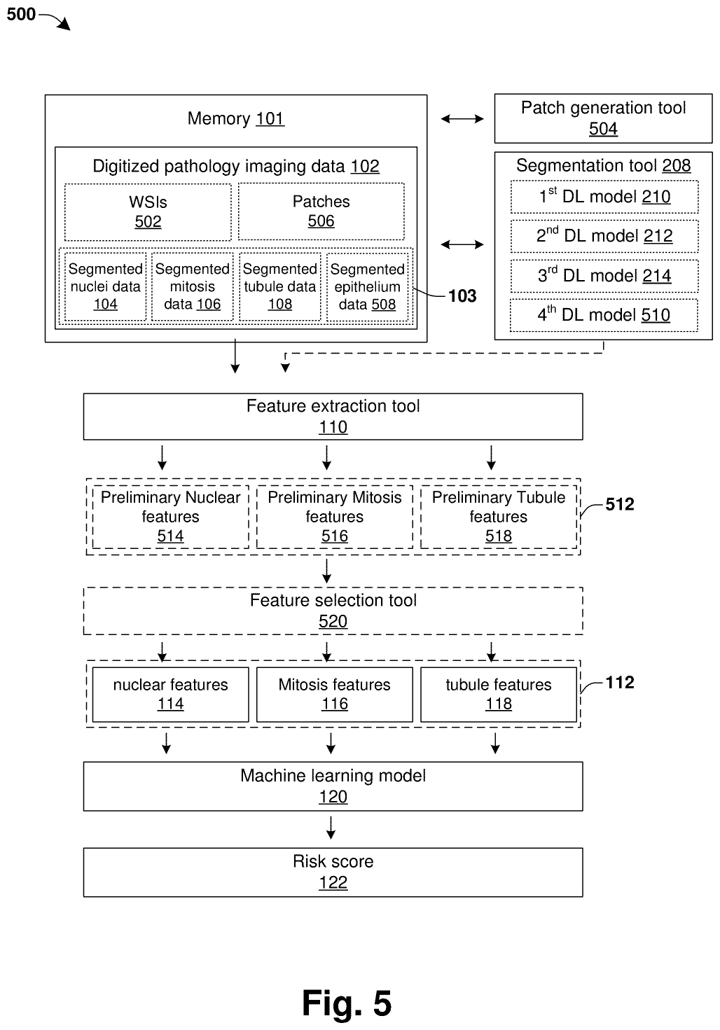

FIG. 5 illustrates a block diagram of some additional embodiments of a disclosed breast cancer risk assessment system 500.

The breast cancer risk assessment system 500 comprises a memory 101 configured to store digitized pathology imaging data 102. In some embodiments, the digitized pathology imaging data 102 may include one or more whole slide images (WSIs) 502 obtained from one or more cancer patients.

In some embodiments, a patch generation tool 504 may be configured to break a digitized image (e.g., WSIs 502) into a plurality of patches 506 (e.g., non-overlapping patches). The plurality of patches 506 may be stored in the memory 101 as part of the digitized pathology imaging data 102. The patch generation tool 504 comprises an additional deep learning model that is configured to identify tumor regions on the digitized pathology imaging data 102 (e.g., WSIs) and to separate the tumor regions into the plurality patches 506 (e.g., tiles). The plurality of patches 506 may subsequently be provided to downstream machine and/or deep learning models to mitigate computational intensity. In some embodiments, the plurality of patches 506 may have a size of approximately 50 pixels×50 pixels, 64 pixels×64 pixels, or other similar values. In some embodiments, patches that contain less than approximately 50% of viable tissue may be discarded.

A segmentation tool 208 is configured to segment the digitized pathology imaging data 102 (e.g., the plurality of patches 506) to generate segmented digitized pathology imaging data 103 including automatically segmented nuclei data 104, automatically segmented mitosis data 106, automatically segmented tubule data 108, and automatically segmented epithelium data 508. In some embodiments, the segmented digitized pathology imaging data 103 comprises and or is binary masks that includes the automatically segmented nuclei data 104, the automatically segmented mitosis data 106, the automatically segmented tubule data 108, and the automatically segmented epithelium data 508.

In some embodiments, the segmentation tool 208 may comprise a plurality of deep learning segmentation models configured to segment the digitized pathology imaging data 102. In some embodiments, the segmentation tool 208 may comprise a first deep learning segmentation model 210 that is configured to operate upon the digitized pathology imaging data 102 to generate the automatically segmented nuclei data 104, a second deep learning segmentation model 212 that is configured to operate upon the digitized pathology imaging data 102 to generate the automatically segmented mitosis data 106, a third deep learning segmentation model 214 that is configured to operate upon the digitized pathology imaging data 102 to generate the automatically segmented tubule data 108, and a fourth deep learning segmentation model 510 that is configured to operate upon the digitized pathology imaging data 102 to generate the automatically segmented epithelium data 508.

A feature extraction tool 110 is configured to extract a plurality of features 112 from the segmented digitized pathology imaging data 103. In some embodiments, the feature extraction tool 110 may be configured to extract a plurality of preliminary features 512 from the segmented digitized pathology imaging data 103. In some embodiments, the plurality of preliminary features 512 include preliminary nuclear features 514, preliminary mitosis features 516, and preliminary tubule features 518. From the plurality of preliminary features 512, a feature selection tool 520 may identify prognostic features that are most prognostic of an outcome (e.g., most prognostic of disease-free survival). After training of the breast cancer risk assessment system 400, the plurality of features 112 that are extracted from the automatically segmented digitized pathology imaging data 103 may be the prognostic features. For example, the breast cancer risk assessment system 400 may be trained to identify the prognostic features from the plurality of preliminary features 512 and then may subsequently extract the plurality of features 112 to only or predominantly be the prognostic features.

In some embodiments, the plurality of features 112 may comprise at least one feature from each of the plurality of preliminary nuclear features 514, the plurality of preliminary mitosis features 516, and the plurality of preliminary tubule features 518. For example, the feature extraction tool 110 may be configured to extract 300 features from the segmented digitized pathology imaging data 103 (e.g., 100 features from the segmented nuclei, 100 from the segmented mitosis, and 100 from the segmented tubules). From the 300 features, the feature selection tool 520 is configured to select the 12 most prognostic features (e.g., a 4 most prognostic from each feature category) that are indicative of risk of recurrence and/or disease-free survival.

The plurality of features 112 include a plurality of nuclear features 114 (e.g., nuclear morphology features), a plurality of mitosis features 116 (e.g., mitosis activity features), and a plurality of tubule features 118 (e.g., tubule formation features). In some embodiments, the feature extraction tool 110 is configured to extract the plurality of nuclear features 114 from the automatically segmented nuclei data 104, the plurality of mitosis features 116 from the automatically segmented mitosis data 106, and the plurality of tubule features 118 from the automatically segmented tubule data 108 and the automatically segmented epithelium data 508. In some embodiments, the feature extraction tool 110 may extract features from each individual patch (e.g., tiles). Patient-level features may then be calculated by aggregating (e.g., using a statistical measure such as mean, median, max, sum, standard deviation, skewness, kurtosis, histogram entropy, and/or approximate entropy) the features across the plurality of patches 506 to form one or more of the plurality of features 112.

In some embodiments, the plurality of nuclear features 114 may comprise features that quantify a nuclear histomorphology of each digitized pathology image, including global graph, shape, cell cluster graph (CCG) features, cell orientation entropy (CORE) features, and Haralick texture feature families. The global graph and CCG feature families, respectively, describe a global and local spatial distribution of nuclei. The shape features capture nuclear boundary properties, such as smoothness and elongation. The CORE features quantitatively measure a disorder degree of nuclear orientations. The Haralick texture features characterize chromosome patterns within nuclei. In some embodiments, the plurality of mitosis features 116 include multiple statistical measurements of the mitotic count, a ratio of mitotic count to epithelial nuclei count, a ratio of mitotic count to blue-ratio nuclei count, and a ratio of mitotic count to nuclei count (e.g., over all extracted tiles across a WSI), a proportion of tiles presenting a specific mitotic density within a WSI, and a quantitative proliferation score calculated by simulating mitosis prevalence assessment in clinical practice. In some embodiments, the plurality of tubule features 118 include various statistical summaries of tubule ratio metrics on all tiles across a WSI of each cancer patient (e.g., a ratio of tubule nuclei count to non-tubule nuclei count, a ratio of tubule nuclei count to epithelium nuclei count, and/or a ratio of tubule nuclei count to nuclei count) as well as a number of tiles falling between different tubule ratio intervals.

A machine learning model 120 is configured to operate upon the plurality of features 112 to generate a risk score 122. In some embodiments, the machine learning model 120 may generate the risk score 122 based on weightings of the plurality of features 112.

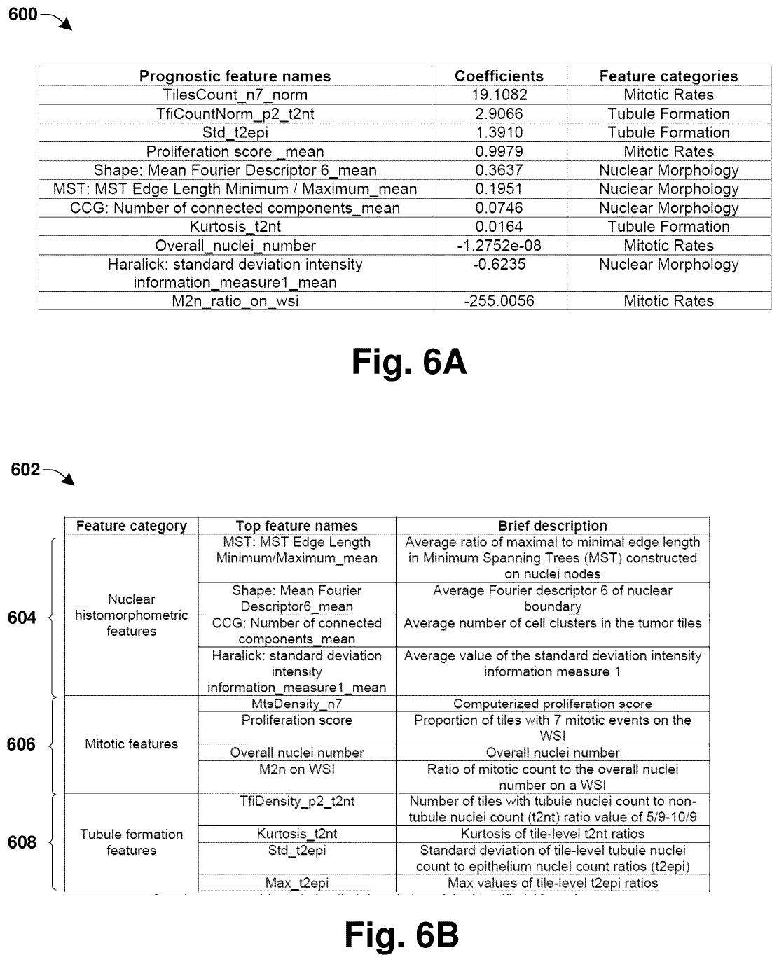

FIG. 6A illustrates a table 600 showing exemplary nuclear features, mitotic features, and tubule features along with associated coefficients assigned by a prognostic Cox regression model.

The table 600 comprises features along with associated coefficients. The coefficients are determined by a feature selection tool (e.g., feature selection tool 520 of FIG. 5) and correlate to a prognostic value of a feature. The higher the absolute value of the coefficient, the more prognostic the feature is to an outcome (e.g., disease-free survival). For example, a ratio of mitosis count to nuclei count (e.g., M2n_ratio_on_wsi) has a coefficient with a higher absolute value than an overall nuclei number, thereby indicating that the ratio of mitosis count to nuclei count is more prognostic than the overall nuclei number.

FIG. 6B illustrates a table 602 showing exemplary nuclear features, mitotic features, and tubule features that have been determined to be prognostic. It will be appreciated that the features shown in table 602 are not limiting of prognostic features but rather are merely examples of prognostic features that may be used by the disclosed cancer risk assessment system.

As shown in table 602, the nuclear features 604 may comprise an average ratio of maximal to minimal edge length in minimum spanning trees constructed on nuclei nodes, an average Fourier descriptor of nuclear boundary, an average number of cell clusters in tumor tiles, and an average value of a standard deviation intensity. The mitosis features 606 may comprise a computerized proliferation score, a proportion of tiles with 7 mitotic events on a whole slide image, an overall nuclei number and a ratio of mitotic count to overall nuclei number on a whole slide image. The tubule features 608 may comprise a number of tiles with tubule nuclei count to non-tubule nuclei count (t2nt) ratio value of 5/9 to 10/9, a Kurtosis of tile level tnt2 ratios, a standard deviation of tile-level tubule nuclei count to epithelium nuclei count ratios (t2epi), and maximum values of tile-level t2epi ratios.

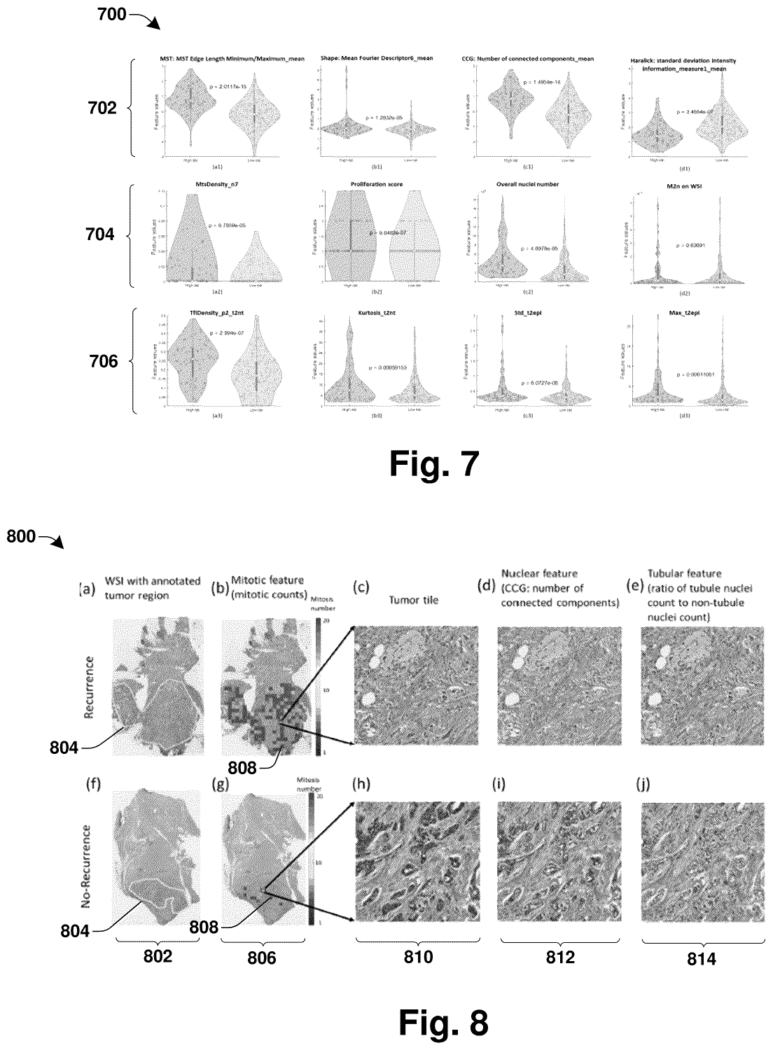

FIG. 7 illustrates exemplary violin graphs 700 showing features values of different nuclear, mitotic, and tubule features for high-risk and low-risk patients.

The violin graphs 700 are illustrated for the features shown in FIG. 6B between high-risk and low-risk groups predicted by the disclosed breast cancer risk assessment system. The first row 702 corresponds to four nuclear features: MST: MST Edge Length Minimum/Maximum_mean, Shape: Mean Fourier Descriptor 6_mean, CCG: Number of connected components mean, and Haralick: standard deviation intensity information_measure1_mean. The second row 704 shows four mitotic features: MtsDensity_n7, Proliferation score, Overall nuclei number, and M2n_on_wsi. The third row 706 displays four tubule features: TfiDensity_p2_t2nt, Kurtosis_t2nt, Std_t2epi, and Max_t2epi. Among the 12 features identified in the violin graphs 700, 11 of them are significantly discriminative (p<0.001) between the two risk groups. Therefore, the violin graphs 700 further illustrate that nuclear, mitotic, and tubule features are able to be used by a disclosed cancer risk assessment system to generate a highly prognostic risk score.

FIG. 8 illustrates exemplary pathology images 800 showing exemplary features nuclear, mitotic, and tubule features.

The pathology images 800 are shown for cancer patients that experienced recurrence and no-recurrence. A first column 802 of the exemplary pathology images 800 shows original WSIs with pathologist-annotated tumor regions 804. A second column 806 illustrates a distribution of mitotic counts 808 on the original WSIs with warmer color in the scale bar indicating a higher mitosis number. The third column 810 is a magnified view of a tumor tile. The fourth column 812 demonstrates an identified nuclear feature, which quantifies a number of connected nuclei clusters (connected in green line). The fifth column 814 shows the tubule feature of a “ratio of tubule nuclei count to non-tubule nuclei count” with tubule nuclei highlighted in cyan. As can be seen by the exemplary pathology images 800, cancer patients that experienced recurrence (e.g., who did not have disease-free survival) tended to have fewer mitotic events, fewer connected nuclear clusters, and a higher proportion of tubule nuclei in relation to cancer patients who did not experience recurrence, thereby further illustrating that nuclear, mitotic, and tubule features are able to be used by a disclosed cancer risk assessment system to generate a highly prognostic risk score.

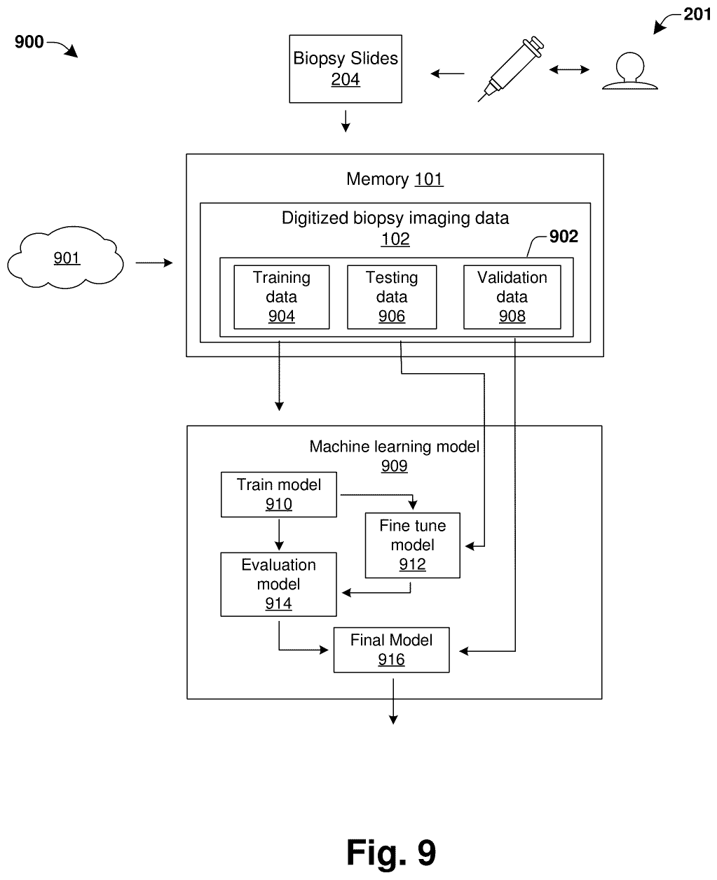

FIG. 9 shows a block diagram 900 illustrating exemplary training of a disclosed breast cancer risk assessment system.

As shown in block diagram 900 of FIG. 9, a memory 101 is configured to store digitized pathology imaging data 102 comprising one or more data sets 902. The one or more data sets 902 may include training data 904 and testing data 906. In some additional embodiments, the one or more data sets 902 may further include validation data 908. The digitized pathology imaging data 102 may be received from digitized pathology samples taken from a cancer patient 201 and/or downloaded from an online database 901 (e.g., an online archive).

The training data 904, the testing data 906, and/or the validation data 908 may be used to train and validate a downstream machine learning model 909. In various embodiments, the machine learning model 909 may comprise one or more of a segmentation tool (e.g., deep learning segmentation models 210, 212, and/or 214), a machine learning model (e.g., machine learning model 120), and/or the like. The training data 904 may be used to train initial versions 910 of the machine learning model 909. The initial versions 910 of the machine learning model 909 may be subsequently fine-tuned 912 using the testing data 906 to generate one or more evaluation models 914. The validation data 908 may then be used to generate a final version 916 of the machine learning model 909 from the one or more evaluation models 914.

In some embodiments, the training data 904, the testing data 906, and/or the validation data 908 may comprise different racial and/or ethic compositions. For example, the training data 904 may comprise a white dominated data set, while the validation data 908 may comprise a south Asian women dominated data set. By having racially and/or ethnically diverse training data 904, testing data 906, and/or validation data 908, the disclosed deep learning segmentation models can be trained to reduce racial and/or ethnic bias in results.

In some embodiments, the training data 904 may comprise different data sets configured to train different machine learning models. For example, the training data 904 may comprise a first training set configured to train a first deep learning segmentation model (e.g., first deep learning segmentation model 210), a second training set configured to train a second deep learning segmentation model (e.g., second deep learning segmentation model 212), a third training set configured to train a third deep learning segmentation model (e.g., third deep learning segmentation model 214), and a fourth training set configured to train a fourth deep learning segmentation model (e.g., fourth deep learning segmentation model 510).

In some embodiments, the first training set used to train the first deep learning segmentation model (e.g., to identify nuclei) may comprise a nuclear annotated dataset comprising 30 images and around 22,000 carefully annotated nuclear boundaries. The inherent diversity of nuclei appearance in the dataset may allow for training a robust model. Image patches corresponding to 256×256 pixels may be extracted from the images within the dataset at ×40 magnification and fed into the first deep learning segmentation model during training.

In some embodiments, the second training set used to train the second deep learning segmentation model (e.g., to identify mitosis) may comprise 550 annotated mitoses in 311 images in size 2000×2000 pixels at 40× magnification from 12 IBC cohorts. Considering the high inter-observer variability for mitosis annotation, a highly experienced board-certified anatomic pathologist may perform mitosis annotation to ensure a quality of ground truth. Small patches (64×64 pixels) may be extracted centering around a mitosis/non-mitosis nuclear centroid as the training set for training the second deep learning segmentation model.

In some embodiments, a training process of the second deep learning segmentation model may include patch extraction, model training, and model refining. Patch extraction may be performed by converting RGB H&E images to gray-scale blue-ratio images, where a higher pixel value indicates higher intensity in blue channel relative to the red and green channels. The high blue-ratio pixels, which are evident to capture candidate mitosis, may be retained to constitute training patches for computational efficiency. To address a huge class imbalance in the training set due to a sparsity of positive (mitotic) pixels, a negative class may be hypo-sampled by random subsampling and positive class size may be augmented by expanding each annotated mitotic centroid into a 9-pixel radius circle so as to extract multiple mitotic patches from one single mitosis annotation. Model training may be performed by dividing the mitotic image dataset into three subsets (e.g., a training subset containing 279 images of size 2000×2000 pixels with 499,194 patches of size 64×64 pixels extracted, in which 23.6% are positive and 76.4% negative; a validation subset of 56 images with 127,645 patches; and a test subset consisting of 32 images with 55,092 patches). The model may be trained using weighted cross-entropy loss in conjunction with an Adam optimizer with image data augmentation for training size enhancement. A model yielding the highest accuracy on a validation set may be selected for additional model refining. The model refining further reduces false-positive detection, by being fine-tuned with an updated training set by randomly substituting 90% of negative patches with all false-positive patches classified by the initially trained model. The optimal refined model may be locked down. In some embodiments, the optimal refined model may yield a balanced accuracy of 0.778 and F1 score of 0.54 on the test set.

In some embodiments, the third training set used to train the third deep learning segmentation model (e.g., to identify tubule regions) may comprise image patches corresponding to n=307 early-stage breast cancers that were 2000×2000 pixels in size and at 40× magnification. The image patches may be randomly extracted from manually annotated tumor regions on digitized slides. The breast tubule structures in each patch may be carefully manually delineated by an experienced pathologist. Patches of size 256×256 pixels may be extracted from the images and augmented to form a dataset of over 1.2 billion patches for training of the third deep learning segmentation model.

In some embodiments, a training process of the third deep learning segmentation model may be performed using twenty tumor tiles randomly selected from another 20 WSI, respectively, with tubule masks overlaid on the top for visual evaluation by an experienced pathologist. One of four grades (e.g., Excellent, Good, Fair, Poor) may be assigned to each tile by the pathologist based on visual examination of machine performance for tubule detection on the tile. The reference accuracy for each category as assigned by the pathologist was: Excellent: >90%, Good: 80%-90%, Fair: 70%-80%, Poor: <70%.” 30% of tiles were ranked as “Excellent”, 45% of tiles “Good”, and 25% of tiles “Fair”.

In some embodiments, the fourth training set used to train the fourth deep learning segmentation model (e.g., to identify epithelium) may comprise manual annotations of epithelium on 200 digital pathology images (e.g., respective having 512×512 pixels) at 10× magnification. In some embodiments, training may be performed using ten tumor tiles randomly selected from 10 WSI, respectively, with tubule masks overlaid on the top for visual evaluation by a pathologist. Each tile was assigned one of four ranking grades (e.g., Excellent, Good, Fair, Poor). The reference accuracy for ranking system is the same with the one used for tubule detection assessment.

In some embodiments, the one or more data sets 902 may be subject to one or more inclusion constraints and/or one or more exclusion constraints. For example, the training data may be selected to exclude images from cancer patients with no H&E stained tumor WSI, clinicopathological data, and/or outcome information; to exclude images from cancer patients with no ODx score; to exclude images from cancer patients that have less than 5 year follow-ups, to exclude images from cancer patients that are not ER+ and LN− IBC, to exclude images from cancer patients with no tumor masks annotated by pathologies; and/or the like.

In some additional embodiments, an optimal risk score threshold may be generated from the training data 904 to dichotomize continuous risk scores into binary high/low-risk categories. In some embodiments, risk scores may be first sorted in descending order across all patients in the training data 904. Subsequently, an average value of each pair of risk scores adjacent to each other may be calculated to constitute a set of candidate risk thresholds. The candidate risk-thresholds set may be further narrowed by trimming elements with extreme values from both ends. In the traversal search process, every candidate risk threshold may be applied to categorize the cancer patients into high-risk or low-risk recurrence groups with a corresponding log-rank p-value and Hazard Ratio (HR) calculated. A risk-threshold yielding a maximal HR may be selected as the optimal risk score threshold for a disclosed breast cancer risk assessment system.

In some embodiments, the optimal risk score threshold may be validated on two independent testing data sets. In some embodiments, a continuous risk score may be calculated for each cancer patient on the two independent testing data sets using the feature coefficients estimated from the training data 904. The cancer patients are then classified into a binary high (e.g., risk score >θₒₚₜ) versus low (e.g., risk score ≤θₒₚₜ) risk category of recurrence by applying θₒₚₜ identified from the training data set.

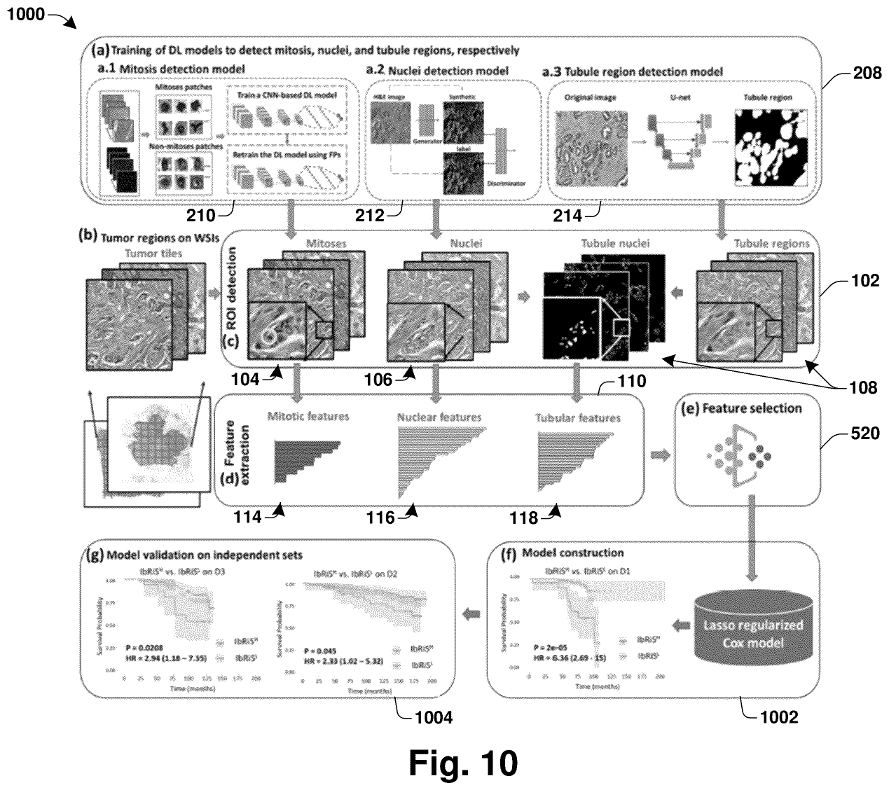

FIG. 10 illustrates a block diagram of some additional embodiments of a disclosed breast cancer risk assessment system 1000.

The breast cancer risk assessment system 1000 comprises a segmentation tool 208 comprising a plurality of deep learning segmentation models 210-214 configured to segment digitized pathology imaging data to generate automatically segmented nuclei data 104, automatically segmented mitosis data 106, and automatically segmented tubule data 108 within a digitized pathology image of a cancer patient. In some embodiments, the plurality of deep learning segmentation models 210-214 may comprise a first deep learning segmentation model 210 that is configured to operate upon the digitized pathology imaging data to generate the automatically segmented nuclei data 104, a second deep learning segmentation model 212 that is configured to operate upon the digitized pathology imaging data to generate the automatically segmented mitosis data 106, and a third deep learning segmentation model 214 that is configured to operate upon the digitized pathology imaging data to generate the automatically segmented tubule data 108.

In some embodiments, the first deep learning segmentation model 210 may comprise a Pixel2Pixel cGAN (conditional general adversarial network) configured to automatically segment the digitized pathology imaging data to generate the automatically segmented nuclei data 104. The Pixel2pixel GAN is an extension of a GAN with both a generator and a discriminator being conditioned on auxiliary image information. The cGAN model may adopt an end-to-end U-Net network as a generator and a multi-layer convolutional network as a discriminator, both of which may be formed based on Convolution-BatchNorm-ReLu modules. Feature matching loss may be additionally added in the standard cGAN loss function to improve a stability of model training by regulating the generator to generate data that matches a distribution of real data.

In some embodiments, the second deep learning segmentation model 212 may comprise a CNN (convolutional neural network) trained to detect mitotic events on the digitized pathology imaging data (e.g., H&E-stained WSIs).

In some embodiments, the third deep learning segmentation model 214 may comprise a U-Net (e.g., a five-layer end to end U-NET) configured to automatically segment tubule regions (e.g., tubules) in breast cancer histopathological images. The third deep learning model may be implemented with Adam optimizer and built with Convolution-BatchNorm-ReLu modules. The combined edge and class weight-based cross-entropy may be employed as the loss function to respectively handle edge detection and class imbalance issues.

In addition, a fourth deep learning model (not shown) may comprise an epithelium segmentation model trained to identify epithelial nuclei for subsequent mitosis ratio calculation. The model used for epithelium detection was of the same architecture as the first deep learning segmentation model 210.

The feature extraction tool 110 is configured to extract nuclear features 114 from the automatically segmented nuclei data 104. In some embodiments, the nuclear features 114 may comprise one or more of nuclear shape features, nuclear texture features, cell orientation entropy (CORE) features, cell cluster graph (CCG) features, and global graph features. The nuclear shape features comprise features that describe information relating to nuclear boundary such as shape irregularity. The nuclear texture features (e.g., Haralick features) comprise features that evaluate heterogeneity patterns relating to chromatin arrangement within each nucleus. The cell orientation entropy (CORE) features comprise features that quantitatively measure a disorder of nuclear orientation within local neighborhoods. The cell cluster graph (CCG) features comprise features that characterize local spatial architecture by constructing sub-graphs on nuclear nodes in local tumor neighborhood extracting features such as cell radius, connectivity, and eccentricity. The global graph features comprise features that describe nuclear architecture by taking each nucleus as a node and connecting nodes via Voronoi diagrams, Delaunay triangles, and minimum spanning trees. In some embodiments, the feature extraction tool 110 is configured to extract 242 nuclear histomorphometric features to quantify the nuclear histomorphology of each WSI.

The feature extraction tool 110 is further configured to extract mitosis features 116, from the automatically segmented mitosis data 106, based on detected mitoses to describe mitosis prevalence status. In some embodiments, the mitosis features 116 may comprise one or more of a mitosis count, mitosis count ratios, a mitosis density Vector, and a proliferation score. The mitosis count comprises features that describe patient-level statistics (e.g., mean, median, max, standard deviation, skewness, and kurtosis) calculated on tile-level mitotic counts for each patient. The mitosis count ratios comprise features that describe patient-level statistics (e.g., mean, median, max, standard deviation, skewness, and kurtosis) calculated on ratios of mitotic count to nuclei count, blue-ratio nuclei count, and epithelium nuclei count on the tile level, respectively. Moreover, mitotic event, detected nuclei, epithelium nuclei, highlighted blue-ratio nuclei may also be accumulatively counted across a WSI to calculate ratios of accumulated mitotic count to other three accumulated counts. The mitosis density vector comprises features that describe a mitotic density vector containing 11-dimensional descriptors. Each bin of the vector calculated a proportion of tiles with n (nϵ0, 1 . . . 9, and n≥10) mitotic events on a WSI, respectively. The histogram entropy and approximate entropy of mitotic density vector also serve as part of the patient-level mitotic features. The proliferation score comprises features that comply with the clinical criteria for tumor proliferation score assignment, by automatically calculating a proliferation score of 1, 2 or 3 was in each WSI. The proliferation score may be calculated on each WSI by simulating the clinical mitosis grading scheme (tumor proliferation score 1 corresponds to a mitotic count of 0-7 per 10 high-power fields (HPFs), score 2 corresponds to 8-15 mitoses count, and score 3 corresponds to ≥16 mitotic count). In some embodiments, a patient-level proliferation score feature may be calculated, as follows:

$${M}_{10 HPFs} = 2 \times \frac{{\sum }_{i = 1}^{T} {M}_{i}}{T} = 2 {M}_{mean} \qquad ( 1 )$$

$${fscore}_{mean} = \{ \begin{matrix}1 , & {M}_{10 HPFs} ∈ [ 0 , 7 ] \\ 2 , & {M}_{10 HPFs} ∈ [ 8 , 15 ] \\ 3 , & {M}_{10 HPFs} \geq 16\end{matrix} \qquad ( 2 )$$

where Mi is a number of mitotic events on an ith tile, T is a number of tiles containing mitotic event in a WSI, Mₘₑₐₙ is an average mitotic count per tile, and M_{10 HPFs} is an average mitotic count per 10 high-power fields (HPFs).

The feature extraction tool 110 is further configured to extract tubule features 118 from the automatically segmented tubule data 108. In some embodiments, the tubule features 118 may comprise one or more of tubule nucleus ratios and tubule ratio distribution vectors. In some embodiments, the tubule nucleus ratios comprise one or more of three tubule ratios, including tubule nuclei count to non-tubule nuclei count, tubule nuclei count to epithelium nuclei count, and tubule nuclei count to nuclei count were calculated at a tile level. Subsequently, eight statistical summaries (mean, median, max, standard deviation, skewness, kurtosis, histogram, entropy, and approximate entropy) may be calculated on three tile-level features to generate 24 patient-level tubule nucleus ratio features. Total tubule nuclei count, overall nuclei count, and overall epithelial nuclei count in a WSI were also calculated. In some embodiments, tubule ratio distribution vectors may comprise a 10-dimensional vector calculated respectively for three tile-level tubule ratio features. Each of the vector bins may count the number of tiles with ratio values of 0-5/9, 5/9-10/9, . . . , 35/9-40/9, 40/9-5, and >5 for the ratio of tubule nuclei count to the non-tubule nuclei count and the ratio of tubule nuclei count to the epithelium nuclei count, ratio values of 0-0.1, 0.1-0.2, . . . , 0.8-0.9, 0.9-1 for the ratio of tubule nuclei count to the nuclei count. In some embodiments, a total of 56 tubule features may be extracted to measure tubule formation based on segmented tubule masks. The features comprise various statistical summaries of tubule ratio metrics on all the tiles across the WSI of each patient (e.g., a ratio of tubule nuclei count to non-tubule nuclei count, a ratio of tubule nuclei count to epithelium nuclei count, and a ratio of tubule nuclei count to nuclei count) as well as a number of tiles falling between different tubule ratio intervals.

In some embodiments, the feature extraction tool 110 is configured to extract a total 343 features (e.g., 242 nuclear pleomorphism features, 45 mitotic count features, and 56 tubule formation features).

A feature selection tool 520 is configured to operate upon the extracted features to perform a feature selection that identifies prognostic features that are important predictors of disease-free survival (DFS). In some embodiments, the feature selection tool 520 may comprise a Cox proportional hazards regression model, regularized by Least Absolute Shrinkage and Selection Operator (LASSO). In some embodiments, to keep a balance among the three feature categories, the Cox regression model may be formed to identify prognostic features associated with DFS separately on each of the three categories. For example, the Cox regression model may be formed to identify a top four prognostic features associated with DFS separately on each of the three categories. The total number of top features (e.g., 12 features) for inclusion within the model may be determined to be approximately 10% of a number of patients within a training set.

Following feature identification, a final LASSO regularized Cox regression model may be used to compute coefficients for each of the features. In some embodiments, 11 features may be assigned non-zero coefficients as part of inclusion within the disclosed breast cancer risk assessment system 1000 while one feature may be assigned a zero-coefficient value.

In some embodiments, the top identified features may be ensembled to construct a final prognostic Cox regression model 1002 by associating them with patient clinical outcomes. In some embodiments, a prognostic significance of the risk score generated by the disclosed breast cancer assessment system may be independently validated 1004 on cohorts from different institutions. In some embodiments, the prognostic significance of the risk score may be independently validated 1004 on two cohorts comprising a total of 205 patients with ER+ and LN− with the differences between high- and low-risk categories measured by two-sided log-rank test.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device. In some embodiments, the computer-executable instructions may be implemented within a software package, so as to allow a health care professional to utilize the disclosed methods and/or block diagrams through the software package.

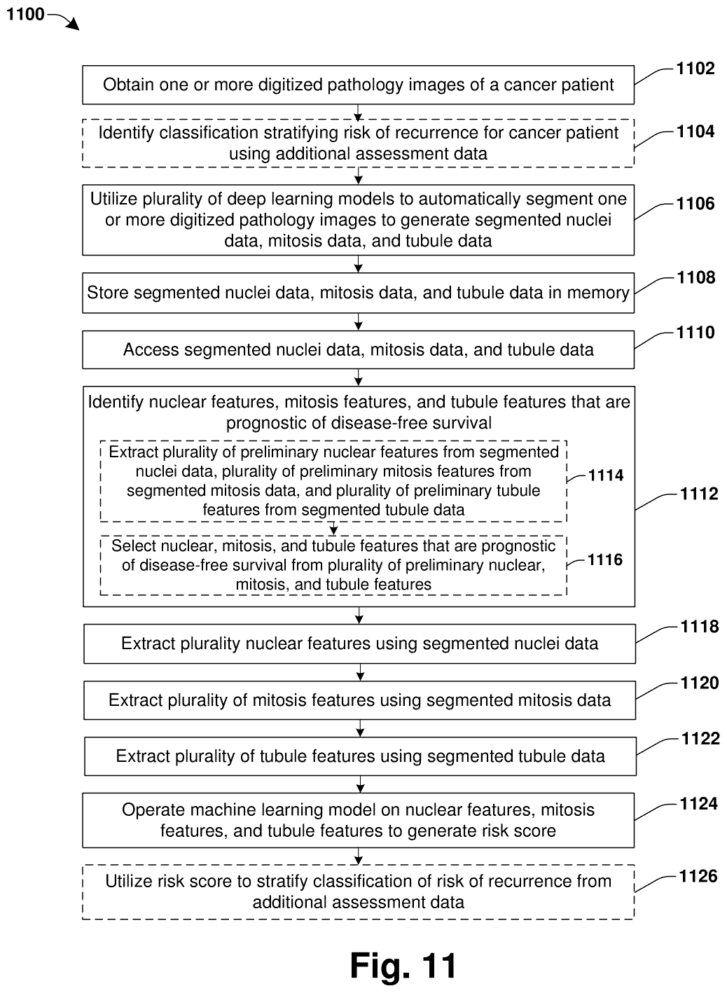

FIG. 11 illustrates a flow diagram of some embodiments of a method 1100 of assessing cancer risk using nuclear, mitotic, and tubule features extracted from digitized pathology imaging data.

While the disclosed method 1100 is illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 1102, one or more digitized pathology images of a cancer patient are obtained. In some embodiments, the cancer patient may be a patient that has estrogen receptor-positive and lymph node-negative invasive breast cancer (ER+ & LN− IBC), estrogen receptor-positive and lymph node-positive invasive breast cancer (ER+ & LN+ IBC), colon cancer, pancreatic cancer, and/or the like.

At act 1104, a classification that stratifies a risk of recurrence for the cancer patient may be identified using additional assessment data, in some embodiments. The additional assessment data may comprise a molecular assay (e.g., an oncotype Dx classification), a Nottingham grading system, and/or the like.

At act 1106, a plurality of deep learning models are utilized to automatically segment the one or more digitized pathology images and to generate segmented nuclei data, mitosis data, and tubule data.

At act 1108, the segmented nuclei data, mitosis data, and tubule data are stored in memory.

At act 1110, the segmented nuclei data, mitosis data, and tubule data are accessed.

At act 1112, nuclear features, mitosis features, and tubule features that are prognostic of disease-free survival are identified. In some embodiments, the nuclear features, mitosis features, and tubule features that are prognostic of disease-free survival are identified according to acts 1114-1116.

At act 1114, a plurality of preliminary nuclear features are extracted from the segmented nuclei data, a plurality of preliminary mitosis features are extracted from the segmented mitosis data, and a plurality of preliminary tubule features are extracted from the segmented tubule data, in some embodiments.

At act 1116, nuclear, mitosis, and tubule features that are prognostic of disease-free survival are selected from the plurality of preliminary nuclear, mitosis, and tubule features, in some embodiments.

At act 1118, the plurality of nuclear features are extracted using the segmented nuclei data.

At act 1120, the plurality of mitosis features are extracted using the segmented mitosis data.

At act 1122, the plurality of tubule features are extracted using the segmented tubule data.

At act 1124, a machine learning model is operated on the nuclear features, the mitosis features, and the tubule features to generate a risk score.

At act 1126, the risk score may be utilized to stratify classifications of the risk of recurrence from the additional assessment data.

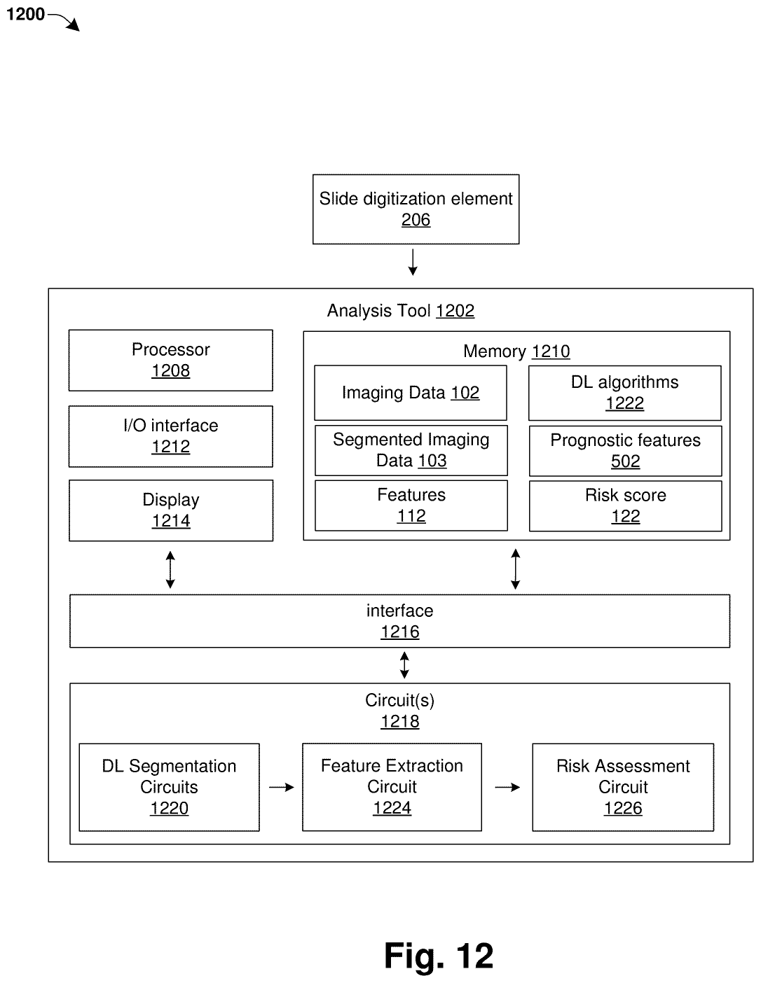

FIG. 12 illustrates a block diagram of some embodiments of a disclosed cancer risk assessment system 1200.

The disclosed cancer risk assessment system 1200 comprises an analysis tool 1202. The analysis tool 1202 is coupled to a slide digitization element 206 that is configured to obtain one or more digitized pathology images (e.g., whole slide images) of one or more tissue samples collected from a cancer patient 201 having cancer (e.g., ER+ & LN− IBC). The slide digitization element 206 is configured to convert biopsy slides to digitized pathology images (e.g., whole slide images), which may be provided to the analysis tool 1202.

The analysis tool 1202 comprises a processor 1208 and a memory 1210. The processor 1208 can, in various embodiments, comprise circuitry such as, but not limited to, one or more single-core or multi-core processors. The processor 1208 can include any combination of general-purpose processors and dedicated processors (e.g., graphics processors, application processors, etc.). The processor(s) 1208 can be coupled with and/or can comprise memory (e.g., memory 1210) or storage and can be configured to execute instructions stored in the memory 1210 or storage to enable various apparatus, applications, or operating systems to perform operations and/or methods discussed herein.

Memory 1210 can be configured to store digitized pathology imaging data 102 comprising digitized pathology images for a plurality of patients having breast cancer (e.g., obtained from the slide digitization element 206). The digitized images may comprise digitized biopsy images having a plurality of pixels, each pixel having an associated intensity. In some additional embodiments, the digitized images may be stored in the memory 1210 as one or more training sets of digitized images, one or more test sets, and/or one or more validation sets of digitized images.

The analysis tool 1202 also comprises an input/output (I/O) interface 1212 (e.g., associated with one or more I/O devices), a display 1214, one or more circuits 1218, and an interface 1216 that connects the processor 1208, the memory 1210, the I/O interface 1212, the display 1214, and the one or more circuits 1218. The I/O interface 1212 can be configured to transfer data between the memory 1210, the processor 1208, the one or more circuits 1218, and external devices.

The one or more circuits 1218 can comprise a plurality of deep learning segmentation circuits 1220 configured to use multiple deep learning algorithms 1222 to generate automatically segmented digitized pathology imaging data 103 that includes automatically segmented nuclei data that identifies segmented nuclei, automatically segmented mitosis data that identifies segmented mitosis, and automatically segmented tubule data that identifies segmented tubule regions. In some embodiments, the plurality of plurality of deep learning segmentation circuits 1220 may comprise hardware components. In other embodiments, the plurality of deep learning segmentation circuits 1220 may comprise software components. In some embodiments, the plurality of deep learning segmentation circuits 1220 may comprise a first deep learning segmentation circuit configured to generate automatically segmented nuclei data, a second deep learning segmentation circuit configured to generate automatically segmented mitosis data, and a third deep learning segmentation circuit configured to generate automatically segmented tubule data.

In some embodiments, the one or more circuits 1218 may further comprise a feature extraction circuit 1224. The feature extraction circuit 1224 is configured to extract a plurality of features 112 from the automatically segmented digitized pathology imaging data 102. The plurality of features 112 include a plurality of nuclear features, a plurality of mitosis features, and a plurality of tubule features.

In some embodiments, the one or more circuits 1218 may further comprise a risk assessment circuit 1226. The risk assessment circuit 1226 is configured to operate upon the plurality of features 112 from to generate a risk score 122.

### Example Use Case:

First, we trained three different deep learning models on H&E-stained Whole Slide Images (WSI) of breast cancer, namely (a1) a Generative Adversarial Network (GAN) for nuclei segmentation, (a2) a deep Convolutional Neural Network (CNN) for mitosis detection, and (a3) a U-Net model for tubule segmentation. Second, based on these computationally derived segmentation/detection masks, we extracted a total of 343 QH features related to nuclear morphology, mitotic count, and tubule formation from the tumor region. Subsequently, we identified the top four prognostic features from each of the three feature categories using a Cox proportional hazards regression model. The top identified features were further ensembled to construct a final prognostic Cox regression model (IbRiS) by associating them with patient clinical outcomes. Finally, we independently validated the prognostic significance of IbRiS on two cohorts from two different institutions, comprising a total of 205 patients with ER+ & LN− IBC. Given the diverse representation of race, tumor grade, and treatment regimen between the training and testing sets, we sought to demonstrate the generalizability of IbRiS for assessing the aggressiveness of breast cancer using computer-extracted histologic features. The prognostic performance of IbRiS was also evaluated within each ODx derived risk category (i.e., low, intermediate, and high).

### Results

### Clinicopathological Variables of the Patient Cohorts

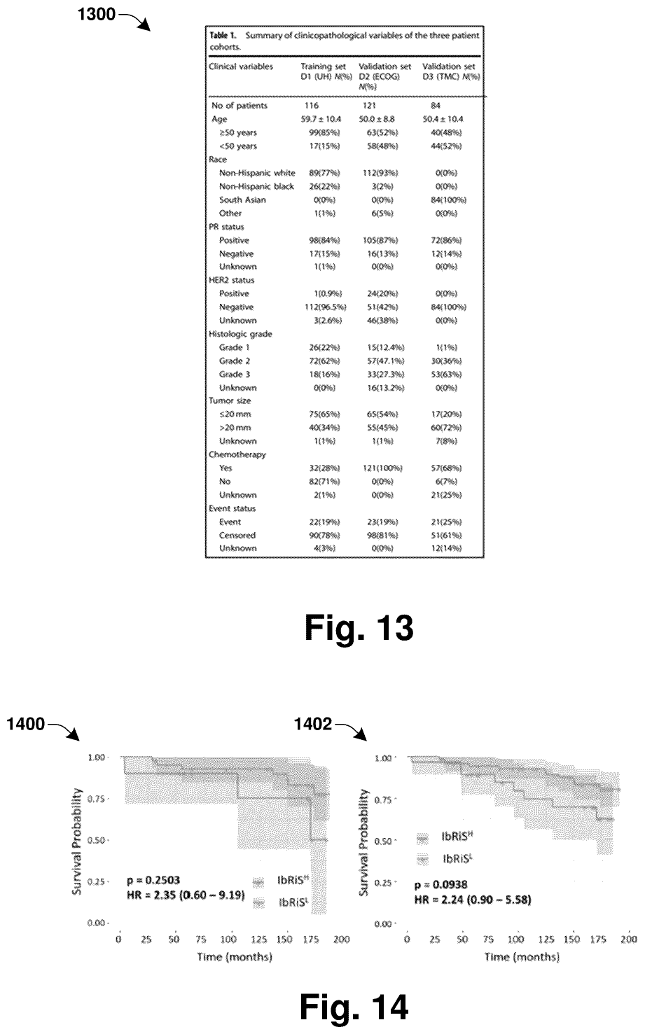

The clinicopathological variables and clinical outcomes of patient cohorts D1, D2, and D3 are provided in table 1300 of FIG. 13. Patients were primarily in their 50s and 60s, and multiple ethnicity groups were included (non-Hispanic white: 62.6%, South Asian: 26.2%, non-Hispanic black: 9%, other: 2.2%). Notably, unlike the non-Hispanic-white-dominated training set D1 and the validation set D2, all patients in the D3 validation set were South Asian women. Approximately 82% of the patients in D1, D2, and D3 were diagnosed as histologic grade 2/grade 3. Particularly, 63% of the patients in D3 were grade 3, much higher than the 16% in D1 and the 27.3% in D2. The vast majority of the patients in D1 and D3 were HER2 negative (HER2−) (except one HER2 positive (HER2+) case in D1) while in D2, 42% patients were HER2−, 20% patients were HER2+, and 38% had unknown HER2 status. Additionally, 65% of all the patients in D₁₊₂₊₃ (D1+D2+D3) were treated with adjuvant chemotherapy (28% in D1, 100% in D2, and 68% in D3). Of note in D1, chemotherapy use was likely guided by the ODX score, unlike the other two cohorts.

### Experiment 1: Model Construction and Validation

A total of 12 prognostic features were obtained by combining the top 4 features identified in each of the three feature categories (i.e., nuclear morphology, mitotic rates, and tubule formation) using a Cox regression model targeting DFS on D1.

A LASSO regularized Cox regression model (IbRiS) was constructed with the 12 identified features correlating to DFS on D1 (n=116) (see Supplementary Table 2 for the non-zero coefficients of the features). A dichotomized risk category was generated from the model as described in Results section. KM survival curves were generated for high (IbRiS^{H}) and low (IbRiS^{L}) risk groups for datasets D1, D2, and D3, respectively, with hazard ratio (HR)=6.36 (95% Confidential Interval (CI)=2.69-15, p=2×10⁻⁵) on D1, HR=2.33 (95% CI=1.02-5.32, p=0.045) on D2, and HR=2.94 (95% CI=1.18-7.35, p=0.0208) on D3.

Patients predicted as high-risk by IbRiS had a significantly worse outcome in terms of DFS than patients in the low-risk group. Notably, the separation of KM curves between IbRiS^{H} and IbRiS^{L} risk groups was more evident beyond the early survival times (˜50 months), which reveals the model's capability in identifying late DFS events. Since 20% of patients in D2 were HER2 positive and 38% had unknown HER2 status, we additionally performed survival analysis of IbRiS on HER2-patients in D2 after excluding the patients with HER2+ or unknown HER2 status (plot 1400 in FIG. 14) as well as on HER2-& HER2 unknown patients in D2 after excluding patients with HER2+ status (plot 1402 in FIG. 14). In both KM curves, the trend that the IbRiS^{H} group had a poorer outcome in terms of DFS was observed, although the survival differentiation is not statistically significant, potentially due to the low number of patients included.

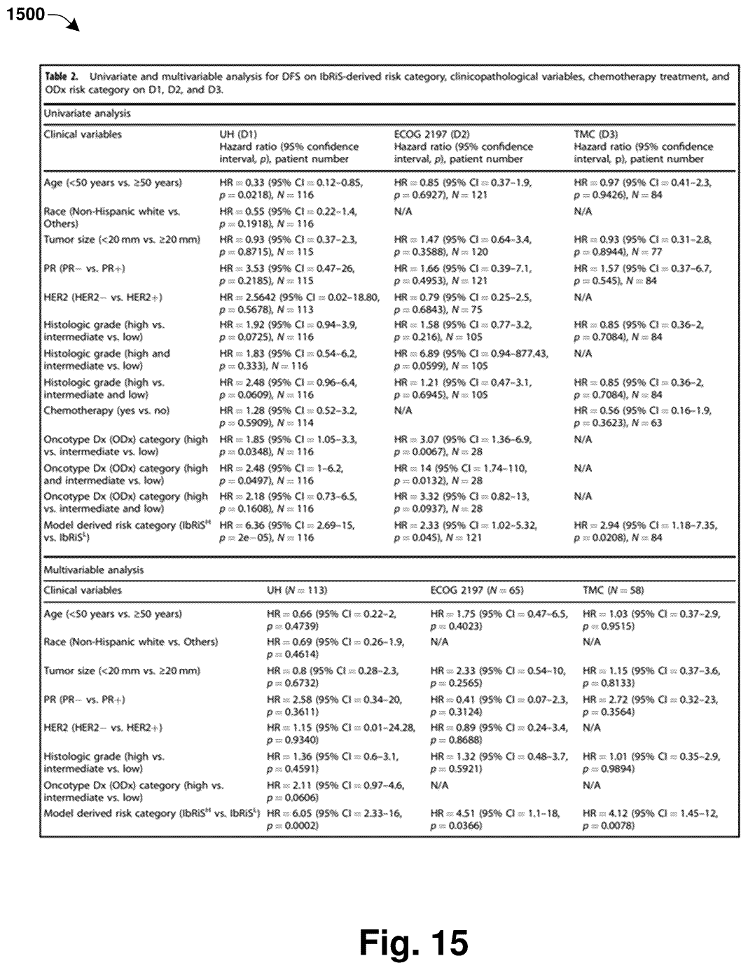

Univariate and multivariable Cox proportional hazards analyses for DFS on IbRiS-derived risk category, clinicopathological variables, chemotherapy treatment, and ODx risk category on D1, D2, and D3 are shown in table 1500 of FIG. 15. On univariate analysis, except for IbRiS-derived risk categories and age on D1, none of the clinicopathological factors was significantly prognostic of DFS on D1, D2, and D3. The patients in IbRiS^{H} had significantly worse DFS compared to those in IbRiS^{L} with HR=6.36 (95% CI=2.69-15, p=2e-05) on D1, HR=2.33 (95% CI=1.02-5.32, p=0.0450) on D2, HR=2.94 (95% CI=1.18-7.35, p=0.0208) on D3). The ODx risk category was significantly prognostic on D1 (HR=2.48, 95% CI=1-6.2, p=0.0497) and D2 (HR=14, 95% CI=1.74-110, p=0.0132) when combining the intermediate and high-risk category into a single group. In multivariable analysis, IbRiS was found to be independently prognostic of DFS in the training set and both independent testing sets with HR=6.05 (95% CI=2.33-16, p=0.0002) on D1, HR=4.51 (95% CI=1.1-18, p=0.0366) on D2, and HR=4.12 (95% CI=1.45-12, p=0.0078) on D3. Note that we excluded the ODx risk category from the multivariable analysis on D2 due to the limited number of patients with ODx scores (23% in D2) available. In order to investigate the interdependency between IbRiS and ODx risk category on D2, Lin's concordance correlation coefficient was calculated with the value of 1 indicating a perfect agreement and −1 representing completely disagreement. The concordance was found to be low between IbRiS (low vs. high-risk group) and ODx test (low & intermediate vs. high ODx risk category: 0.16 (95% CI=−0.21-0.49); low vs. intermediate & high ODx risk category: 0.26 (95% CI=−0.08-0.54)).

### Experiment 2: IbRiS-Derived Risk Category Versus ODx Risk Category

We sought to demonstrate the prognostic ability of IbRiS-derived risk scores within each individual ODx category. ODx scores were available for n=116 patients in D1 and n=28 patients in D2. Patients in the IbRiS^{H} group experienced a higher relapse probability than those classified as IbRiS^{L} in the high ODx categories for both D1 and D2.

Specifically, in the high ODx risk category (D₁₊₂), among the 10 patients predicted as IbRiS^{L}, 9 patients had favorable outcomes (non-DFS event with a median follow-up of approximately 7 years) while among the 7 patients identified as high-risk by IbRiS, 5 of them suffered recurrence/death.

### Experiment 3: IbRiS-Derived Risk Category Versus Histologic Grade

We sought to demonstrate the prognostic ability of IbRiS-derived risk categories in subgroups stratified by pathologist-assigned histologic grades. For the high-grade groups, patients predicted as IbRiS^{H} had significantly worse prognosis than those predicted as IbRiS^{L} for all the three cohorts. Specifically, for the pathologist-assigned high-grade group (D₁₊₂₊₃), 50% of patients identified as IbRiS^{H} suffered from DFS events, while among the patients classified as IbRiS^{L} only 14% recurred/died.

Therefore, the present disclosure relates to a method and apparatus configured to assess a risk of cancer recurrence for a cancer patient based upon a machine learning model trained on a combination of nuclear features, mitosis features, and tubule features.

In some embodiments, the present disclosure relates to a method, the method including accessing segmented digitized pathology imaging data from a cancer patient, the segmented digitized pathology imaging data identifying segmented nuclei, segmented mitosis, and segmented tubule regions; extracting a plurality of nuclear features using the segmented nuclei; extracting a plurality of mitosis features using the segmented mitosis; extracting a plurality of tubule features using the segmented tubule regions; and generating a risk score by operating a machine learning model on the plurality of nuclear features, the plurality of mitosis features, and the plurality of tubule features, the risk score correlating to a risk of recurrence of cancer for the cancer patient

In other embodiments, the present disclosure relates to a non-transitory computer-readable medium storing computer-executable instructions that, when executed, cause a processor to perform operations, including utilizing a first deep learning segmentation model to automatically segment one or more digitized pathology images to identify segmented nuclei, the one or more digitized pathology images corresponding to a cancer patient; utilizing a second deep learning segmentation model to automatically segment the one or more digitized pathology images to identify segmented mitosis; utilizing a third deep learning segmentation model to automatically segment the one or more digitized pathology images to identify segmented tubule regions; extracting a plurality of nuclear features using the segmented nuclei; extracting a plurality of mitosis features using the segmented mitosis; extracting a plurality of tubule features using the segmented tubule regions; and generating a risk score by operating a regression model on the plurality of nuclear features, the plurality of mitosis features, and the plurality of tubule features, the risk score correlating to a risk of recurrence of cancer for the cancer patient.

In yet other embodiments, the present disclosure relates to an apparatus, the apparatus including a memory configured to store segmented digitized pathology imaging data for a cancer patient, the segmented digitized pathology imaging data including segmented nuclei data, segmented mitosis data, and segmented tubule data; a feature extraction tool configured to extract a plurality of nuclear features using the segmented nuclei data, to extract a plurality of mitosis features using the segmented mitosis data, and to extract a plurality of tubule features from the segmented tubule data; and a machine learning model configured to use the plurality of nuclear features, the plurality of mitosis features, and the plurality of tubule features to generate a risk score that correlates to a risk of cancer recurrence for the cancer patient.

Examples herein can include subject matter such as an apparatus, a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
accessing segmented digitized pathology imaging data from a cancer patient, the segmented digitized pathology imaging data identifying segmented nuclei, segmented mitosis, and segmented tubule regions;
extracting a plurality of nuclear features using the segmented nuclei;
extracting a plurality of mitosis features using the segmented mitosis;
extracting a plurality of tubule features using the segmented tubule regions; and
generating a risk score by operating a machine learning model on the plurality of nuclear features, the plurality of mitosis features, and the plurality of tubule features, the risk score correlating to a risk of recurrence of cancer for the cancer patient.

2. The method of claim 1, further comprising:
utilizing a first deep learning segmentation model to automatically segment one or more digitized pathology images to identify the segmented nuclei;
utilizing a second deep learning segmentation model to automatically segment the one or more digitized pathology images to identify the segmented mitosis; and
utilizing a third deep learning segmentation model to automatically segment the one or more digitized pathology images to identify the segmented tubule regions.

3. The method of claim 1,
wherein the segmented digitized pathology imaging data further identifies segmented epithelium; and
wherein the plurality of tubule features are extracted using the segmented tubule regions and the segmented epithelium.

4. The method of claim 1, wherein the cancer patient has estrogen receptor-positive (ER+) and lymph node-negative (LN−) invasive breast cancer or estrogen receptor-positive (ER+) and lymph node-positive (LN+) invasive breast cancer.

5. The method of claim 1, further comprising:
classifying the cancer patient into a risk classification using additional assessment data, wherein the risk score further stratifies the risk classification.

6. The method of claim 5, wherein the additional assessment data comprises an oncotype Dx classification.

7. The method of claim 5, wherein the additional assessment data comprises an Nottingham grading system (NGS) classification.

8. The method of claim 1, wherein the plurality of nuclear features comprise one or more of an average ratio of maximal to minimal edge length in minimum spanning trees constructed on nuclei nodes, an average Fourier descriptor of nuclear boundary, an average number of cell clusters in tumor tiles, and an average value of a standard deviation intensity.

9. The method of claim 1, wherein the plurality of mitosis features comprise one or more of a computerized proliferation score, a proportion of tiles with 7 mitotic events on a whole slide image, an overall nuclei number, and a ratio of mitotic count to overall nuclei number on a whole slide image.

10. The method of claim 1, wherein the plurality of tubule features comprise one or more of a number of tiles with tubule nuclei count to non-tubule nuclei count (t2nt) ratio value of 5/9 to 10/9, a Kurtosis of tile level tnt2 ratios, a standard deviation of tile-level tubule nuclei count to epithelium nuclei count ratios (t2epi), and maximum values of tile-level t2epi ratios.

11. A non-transitory computer-readable medium storing computer-executable instructions that, when executed, cause a processor to perform operations, comprising:
utilizing a first deep learning segmentation model to automatically segment one or more digitized pathology images to identify segmented nuclei, the one or more digitized pathology images corresponding to a cancer patient;
utilizing a second deep learning segmentation model to automatically segment the one or more digitized pathology images to identify segmented mitosis;
utilizing a third deep learning segmentation model to automatically segment the one or more digitized pathology images to identify segmented tubule regions;
extracting a plurality of nuclear features using the segmented nuclei;
extracting a plurality of mitosis features using the segmented mitosis;
extracting a plurality of tubule features using the segmented tubule regions; and
generating a risk score by operating a regression model on the plurality of nuclear features, the plurality of mitosis features, and the plurality of tubule features, the risk score correlating to a risk of recurrence of cancer for the cancer patient.

12. The non-transitory computer-readable medium of claim 11, wherein the first deep learning segmentation model is a conditional general adversarial network (GAN), the second deep learning segmentation model is a convolutional neural network (CNN), and the third deep learning segmentation model is a U-Net.

13. The non-transitory computer-readable medium of claim 11, wherein the plurality of nuclear features comprise one or more of nuclear shape features, nuclear texture features, cell orientation entropy (CORE) features, cell cluster graph (CCG) features, and global graph features.

14. The non-transitory computer-readable medium of claim 11, wherein the plurality of mitosis features comprise one or more of a mitosis count, mitosis count ratios, a mitosis density vector, and a proliferation score.

15. The non-transitory computer-readable medium of claim 11, wherein the plurality of tubule features comprise one or more of tubule nucleus ratios and tubule ratio distribution vectors.

16. An apparatus, comprising:
a memory configured to store segmented digitized pathology imaging data for a cancer patient, the segmented digitized pathology imaging data including segmented nuclei data, segmented mitosis data, and segmented tubule data;
a feature extraction tool configured to extract a plurality of nuclear features using the segmented nuclei data, to extract a plurality of mitosis features using the segmented mitosis data, and to extract a plurality of tubule features from the segmented tubule data; and
a machine learning model configured to use the plurality of nuclear features, the plurality of mitosis features, and the plurality of tubule features to generate a risk score that correlates to a risk of cancer recurrence for the cancer patient.

17. The apparatus of claim 16, wherein the cancer patient has estrogen receptor-positive (ER+) and lymph node-negative (LN−) invasive breast cancer, estrogen receptor-positive (ER+) and lymph node-positive (LN+) invasive breast cancer, colon cancer, or pancreatic cancer.

18. The apparatus of claim 16, wherein the plurality of nuclear features comprise one or more of nuclear shape features, nuclear texture features, cell orientation entropy (CORE) features, cell cluster graph (CCG) features, and global graph features.

19. The apparatus of claim 16, wherein the plurality of mitosis features comprise one or more of a mitosis count, mitosis count ratios, a mitosis density vector, and a proliferation score.

20. The apparatus of claim 16, wherein the plurality of tubule features comprise one or more of tubule nucleus ratios and tubule ratio distribution vectors.
